(19) United States
(12) Patent Application Publication
(10) Pub. No.: US 2022/0348873 A1
(43) Pub. Date: Nov. 3, 2022

(54) METHOD FOR INCREASING CONTENT OF NK CELLS USING CULTURE MEDIUM OF IMMUNE CELLS OF HEALTHY PERSON

(71) Applicants: Jeong Chan RA, Chungcheongbuk-do (KR); Sang Kyu WOO, Gyeonggi-do (KR); Sung Ik CHOI, Busan (KR)

(72) Inventors: Jeong Chan RA, Chungcheongbuk-do (KR); Sang Kyu WOO, Gyeonggi-do (KR); Sung Ik CHOI, Busan (KR)

(21) Appl. No.: 17/620,183

(22) PCT Filed: Jun. 12, 2020
(86) PCT No.: PCT/KR2020/007660

§ 371 Date: Dec. 17, 2021

(30) Foreign Application Priority Data
Jun. 17, 2019 (KR) 10-2019-0071257

(51) Int. Cl. C12N 5/0783; A61K 35/17

(52) U.S. Cl. CPC C12N 5/0638; A61K 35/17; C12N 2501/515; C12N 2501/599; C12N 2502/11

(57) ABSTRACT

The present invention relates to a culture method for increasing the content of NK cells. According to the present invention, the growth rate of immune cells including NK cells can be increased not only in cancer patients but also in persons who are genetically susceptible to cancer or elderly people, and an NK cell fraction and cell-killing ability can also be enhanced.

### TECHNICAL FIELD

The present invention relates to a culture method for increasing NK cell content, and more particularly to a method of culturing NK cells including culturing immune cells in a medium containing a normal-person immune-cell cultured solution.

### BACKGROUND ART

Numerous cancer cells are generated every day in the human body, and the generated cancer cells are killed by the body's immune system. The cells that play the most important role in the immune system are natural killer cells (hereinafter, referred to as ‘NK cells’).

NK cells are an important contributor to the innate immune capability of the human body, and importantly serve mainly to eliminate tumor cells and virus-infected cells. The proportion of NK cells present in the body of healthy people is known to be about 5-20%. However, it is known that the proportion of NK cells in cancer patients is lower than in healthy people and also that NK cell efficiency thereof is lowered (J. Chen, J et al., Int. J. Clin. Exp. Pathol. 7:8304, 2014). In cancer patients, the normal immune system is not activated due to this deterioration in the number and function of NK cells, thereby leading to cancer.

Most NK cells in the body exist in an inactivated state under normal conditions. However, in order to use NK cells in practice for therapeutic purposes, activated NK cells are required, so thorough research on activating NK cells from normal blood or from patient blood in which NK cells are inactivated is ongoing.

It has been confirmed that NK cells exhibit high cytotoxicity when activated ex vivo, suggesting the possibility of immune cell therapy using NK cells. There is a report confirming the therapeutic effect of NK cells activated ex vivo through administration after an allogeneic bone marrow transplant to patients with various types of cancer, especially blood cancer such as leukemia (Blood Cells Molecules & Disease, 33: p 261-266, 2004).

Meanwhile, despite the potential use of NK cells described above as a therapeutic agent, the number of NK cells present in the body is not large, so technology for mass production of NK cells while maintaining sufficient efficacy thereof for therapeutic purposes is essential. However, NK cells are not properly mass-proliferated and cultured in vitro. Therefore, technology for amplifying and culturing NK cells to a level useful for practical application is receiving attention, and has been thoroughly studied but has not yet reached a level applicable to clinical practice.

In order to culture NK cells, research has been carried out using not only IL-2, which is conventionally used for T cell proliferation/activation, but also IL-15 (J. Immunol., 167(6):p 3129-3138, 2001; Blood, 106(1): p 158-166, 2005, Korean Patent Publication No. 2009-0121694), LPS (J. Immunol., 165(1): p 139-147, 2000), or the OKT-3 antibody, which stimulates CD3 (Experimental Hematol., 29(1): p 104-113, 2001), but this research has merely found a new proliferation material in a modification and development form for the use of IL-2, which is conventionally used, but does not suggest an innovative proliferation method. In general, when NK cells are cultured using IL-2 or other cytokines and compounds (chemicals), the number of cells is known to increase only to about 3 to 10 times the initial number of NK cells.

Restoration of the performance of NK cells of cancer patients to the performance of NK cells of healthy people and the use of immune cells of healthy donors (non-self) are under study (J. Chen, J et al., Int. J. Clin. Exp. Pathol. 7:8304, 2014; E. G. Iliopoulou et al., Cancer Immunol. Immunother. DOI:10.1007/s00262-010-0904-3, 2010; SR Yoon et al., Bone Marrow Transplant., DOI:10.1038/bmt.2009.304, 2010). However, there are inconveniences such as the addition of many types of interleukins and the selective classification of NK cells in the PBMC stage in the production of these immune cell medicines.

Therefore, the present inventors have made great efforts to develop a method of increasing the number of NK cells more economically and efficiently when culturing NK cells, and thus ascertained that, when a healthy-person immune-cell cultured solution is added during culture of patient-derived immune cells, NK cell content is increased, thereby completing the present invention.

### SUMMARY OF THE INVENTION

It is an object of the present invention to provide a method of increasing NK cell content.

In order to accomplish the above object, the present invention provides a method of increasing NK cell content comprising culturing peripheral blood mononuclear cells (PBMCs) derived from a cancer patient or a normal person in a medium containing a normal-person immune-cell cultured solution and a CD3 antibody.

### BRIEF DESCRIPTION OF DRAWINGS

FIG. 1 is a graph showing the results of comparison of the number of lymphocytes in experimental groups in which immune cells of cancer patients are cultured with the addition of a healthy-person immune-cell cultured solution and a control group (immune cells cultured without the addition of a healthy-person immune-cell cultured solution).

FIG. 2 and Table 1 show the results of FACS on experimental groups in which immune cells of cancer patients are cultured with the addition of the healthy-person immune-cell cultured solution and a control group (immune cells cultured without the addition of the healthy-person immune-cell cultured solution).

FIG. 3 shows results confirming the cytotoxicity of an experimental group in which immune cells of cancer patients cultured with the addition of the healthy-person immune-cell cultured solution are co-cultured with cancer cells and a control group (in which immune cells cultured without the addition of the healthy-person immune-cell cultured solution are co-cultured with cancer cells).

### DETAILED DESCRIPTION AND PREFERRED EMBODIMENTS OF THE INVENTION

Unless otherwise defined, all technical and scientific terms used herein have the same meanings as those typically understood by those skilled in the art to which the present invention belongs. Generally, the nomenclature used herein is well known in the art and is typical.

In the present invention, in order to restore the performance of NK cells of cancer patients to the performance of NK cells of healthy people, many attempts have been made to develop a method of increasing the NK cell content in immune cells. When a cultured solution obtained by culturing immune cells derived from healthy young people is used to culture immune cells derived from cancer patients, it is confirmed that the number of lymphocytes is increased and also that the fraction of NK cells in the immune cells is increased.

Accordingly, the present invention pertains to a method of increasing NK cell content comprising culturing peripheral blood mononuclear cells (PBMCs) derived from a cancer patient or a normal person in a medium containing a normal-person immune-cell cultured solution and a CD3 antibody.

As used herein, the term ‘normal person’ refers to a healthy person who is 16 to 40 years old and does not have a diagnosed disease.

As used herein, the term ‘immune cells’ refer to lymphocyte-based immune cells, and preferably immune cells contained in peripheral blood mononuclear cells (PBMCs).

In the present invention, the ‘immune cells’ may use immune cells resulting from P1 to P3 subculture of peripheral blood mononuclear cells (PBMCs), and preferably use immune cells stimulated with an NK cell proliferation stimulator, in order to stimulate the proliferation of NK cells.

The ‘NK cell proliferation stimulator’ may include, but is not limited to, a CD3 antibody, CD 16 antibody, CD 56 antibody, IL-2, IL-15, LPS, OKT-3 antibody, or the like.

In the present invention, the normal-person immune-cell cultured solution is a cultured solution produced by subjecting peripheral blood mononuclear cells (PBMC) derived from a normal person to P1-P3 culture, and the peripheral blood mononuclear cells (PBMC) are cultured by the addition of a cytokine antibody selected from the group consisting of CD3, CD16, and CD56.

In the present invention, the immune-cell cultured solution is a cell-free cultured solution from which cells are removed.

In the present invention, the immune cells may be immune cells derived from a cancer patient or a normal person.

In the present invention, the immune cells may be characterized in that peripheral blood mononuclear cells (PBMC) derived from a cancer patient or a normal person are cultured in the presence of a CD3 antibody.

In the present invention, the concentration of the normal-person immune-cell cultured solution that is added to the medium is 1 to 50%, preferably 5 to 30%, and more preferably 10 to 20%.

In the present invention, the normal-person immune-cell cultured solution may be added upon P2 culture during the culture of immune cells.

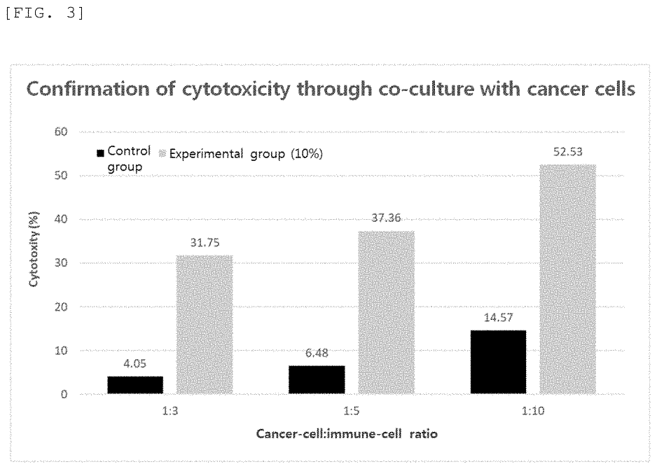

In an embodiment of the present invention, K562 cells, which are a chronic myeloid leukemia cell line targeted by NK cells, and immune cells, cultured by adding 10% of a healthy-person immune-cell cultured solution, were mixed at ratios of 1:3, 1:5 and 1:10 and co-cultured, and the cytotoxicity of the immune cells against the cancer cells was evaluated. Thus, it was confirmed that the cytotoxicity against K562 at respective K562-cell:immune-cell ratios of 1:3, 1:5 and 1:10 was higher in the experimental group added with the healthy-person immune-cell cultured solution than in the control group (FIG. 3).

The NK cells produced by the method according to the present invention and a composition containing the same may be used for the treatment of tumors and infectious diseases. The NK cells produced by the method according to the present invention may be applied to all types of tumors including solid cancer and blood cancer. Unlike blood cancer, solid cancer is cancer resulting from formation of a lump in an organ, and cancer occurring in most organs corresponds to solid cancer. There is no particular limitation as to the types of tumors that may be treated using the NK cells according to the present invention, and examples of such tumors may include, but are not limited to, gastric cancer, liver cancer, lung cancer, colorectal cancer, breast cancer, prostate cancer, ovarian cancer, pancreatic cancer, cervical cancer, thyroid cancer, laryngeal cancer, acute myeloid leukemia, brain tumors, neuroblastoma, retinoblastoma, head and neck cancer, salivary gland cancer, lymphoma, and the like.

The infectious disease used in the present invention is a disease caused by infection with a virus or pathogen, and conceptually includes all diseases that may be transmitted and infected through respiratory, blood, and dermal routes. Non-limiting examples of such infectious diseases include, but are not limited to, hepatitis B and C, human papilloma virus (HPV) infection, cytomegalovirus infection, viral respiratory diseases, influenza, coronavirus infection, and the like.

A better understanding of the present invention may be obtained through the following examples. These examples are merely set forth to illustrate the present invention, and are not to be construed as limiting the scope of the present invention, as will be apparent to those of ordinary skill in the art.

### Example 1: Preparation of Cultured Solution by Culturing Immune Cells of Healthy Young Person

1-1: Isolation of PBMCs (Peripheral Blood Mononuclear Cells)

50 ml of blood from a 25-year-old healthy person without a diagnosed disease was diluted at 1:1 (blood:PBS) using DPBS, 20 ml of the diluted blood was placed in a tube containing 15 ml of Ficoll (GE Healthcare), centrifugation was performed at 400 g for 45 minutes, and the cells distributed in the buffy coat layer were recovered. The cells thus recovered were mixed with 40 ml of PBS and then centrifuged at 400 g for 5 minutes, the supernatant was removed, and the number of isolated PBMCs was counted.

1-2: Preparation of Immune-Cell Cultured Solution

P0 culture: The PBMCs isolated in 1-1 above were evenly divided and cultured using a KBM501 medium (Kohjin Bio, Cat. No. 1625015) in two T75 flasks, and during culture, 0.025% of a CD3 antibody (BD Pharmingen, Cat. No 566685) and a CD56 antibody (BD Pharmingen, Cat. No 559043) were added to each of the two flasks, 0.15% of a CD16 antibody (BD Pharmingen, Cat. No 555404) was added to one flask (CD16+ flask) thereof, and the CD16 antibody was further added on the first day and the third day of culture.

Culture was carried out in a CO₂ incubator (CO₂ concentration: 5.0%) at 37° C., and all subsequent culture was performed under the same conditions. On the second day of culture, a medium supplemented with 10% FBS was added to each flask, and subculture was performed on the fifth day of culture.

P1 culture: The cells recovered after P0 culture were added to each T175 flask. Here, the cultured solution was not discarded but was placed in the T175 flasks (Flask 1: for addition of CD16, Flask 2: not for addition of CD16). As such, 0.05% of a CD3 antibody was further added to each flask and cultured using a KBM501 medium (Kohjin Bio, Cat. No. 1625015). On the first day of P1 culture, a CD56 antibody (BD Pharmingen, Cat. No 559043) was added to each flask, and 0.2% of a CD16 antibody was further added to the CD16+ flask. On the second day of culture, a medium supplemented with 1% FBS was added to each flask. On the third day of culture, 0.12% of the CD56 was further added to each flask, and 0.2% of the CD16 antibody was further added to the CD16+ flask. On the fourth day of culture, a medium supplemented with 1% FBS was further added to each flask, and on the eighth day of culture, the cultured solutions of the two flasks were recovered, combined, and used for NK cell culture.

### Example 2: Culture of NK Cells of Cancer Patient

2-1: Isolation of PBMCs (Peripheral Blood Mononuclear Cells)

50 ml of blood from a 39-year-old brain-tumor-diagnosed patient was diluted at 1:1 (blood:PBS) using DPBS, 20 ml of the diluted blood was placed in a tube containing 15 ml of Ficoll, centrifugation was performed at 400 g for 5 minutes, and the cells distributed in the buffy coat layer were recovered. The cells thus recovered were mixed with 40 ml of PBS and then centrifuged at 400 g for 5 minutes, the supernatant was removed, and the number of isolated PBMC cells was counted.

2-2: NK Cell Culture

P0 culture: The PBMCs isolated in 2-1 above were evenly divided and cultured using a KBM501 medium (Kohjin Bio, Cat. No. 1625015) in two T75 flasks, and during culture, 0.025% of a CD3 antibody (BD Pharmingen, Cat. No 566685) and 0.075% of a CD56 antibody (BD Pharmingen, Cat. No 559043) were added to each of the two flasks, and 0.15% of a CD16 antibody (BD Pharmingen, Cat. No 555404) was added to one flask (CD16+ flask) thereof, and on the first and third days of culture, 0.015% of the CD16 antibody was further added thereto.

Culture was carried out in a CO₂ incubator (CO₂ concentration: 5.0%) at 37° C., and all subsequent culture was performed under the same conditions. On the second day of culture, a medium supplemented with 10% FBS was added to each flask, and subculture was performed on the fifth day of culture.

P1 culture: The cells recovered after P0 culture were added to each T175 flask. As such, the cultured solution was not discarded but was placed in the T175 flasks (Flask 1: for addition of CD16, Flask 2: not for addition of CD16). Here, a KBM501 medium and 0.05% of a CD3 antibody were further added to each flask and cultured. On the first day of P1 culture, 0.012% of a CD56 antibody (BD Pharmingen, Cat. No 559043) was added to each flask, and 0.2% of a CD16 antibody was further added to the CD16+ flask. On the second day of culture, a medium supplemented with 1% FBS was added to each flask. On the third day of culture, 0.12% of the CD56 antibody was further added to each flask, and the CD16 antibody was further added to the CD16+ flask. On the fourth day of culture, a medium supplemented with 1% FBS was further added to each flask, and subculture was performed on the fifth day of culture.

P2 culture: The cells and the cultured solution recovered after P1 culture were added as they were to KBM502B (KOHJIN BIO, Cat. No. 1602502B). Here, 10% FBS and the healthy-person immune-cell cultured solution obtained in Example 1 at concentrations of 10%, 15% and 20% were added thereto, and the cells were cultured for 7 days and then recovered.

### Example 3: Measurement of Total Number of Immune Cells and NK Cell Fraction

As experimental groups, immune cells cultured by the method of Example 2 were used, and as a control group, immune cells cultured without adding a healthy-person immune-cell cultured solution were used.

The number of cancer-patient-derived immune cells was measured using a microscope and a hemocytometer, and the NK cell fraction was determined through FACS assay (BD FACSVerse, BD Bioscience) (FITC-CD3/PE-CD16,56).

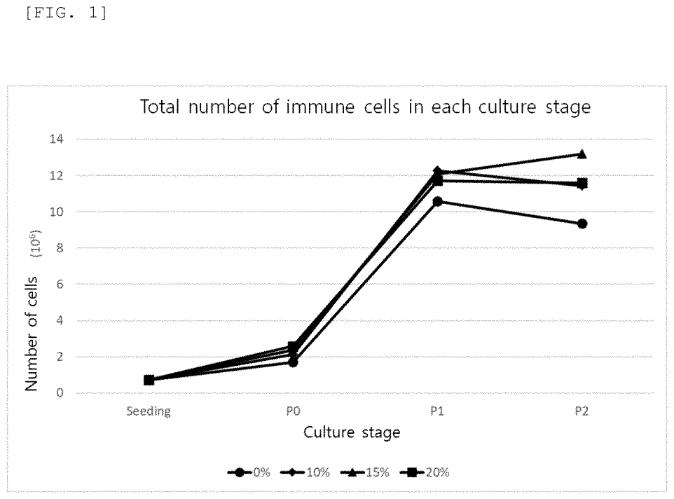

Based on the results thereof, as shown in FIG. 1 and Table 1 below, it was confirmed that the number of cells was higher in all of the experimental groups in which the healthy-person immune-cell cultured solution was added at concentrations of 10%, 15% and 20% and cultured than in the control group.

TABLE 1

Total number of immune cells in each
culture stage (unit: 10⁶)

| | Seeding | P0 | P1 | P2 |
|---|---|---|---|---|
| Control group | 0.72 | 1.70 | 10.59 | 9.36 |
| Experimental | 0.72 | 2.12 | 12.28 | 11.44 |
| group (10%) | | | | |
| Experimental | 0.72 | 2.36 | 12.10 | 13.20 |
| group (15%) | | | | |
| Experimental | 0.72 | 2.58 | 11.72 | 11.60 |
| group (20%) | | | | |

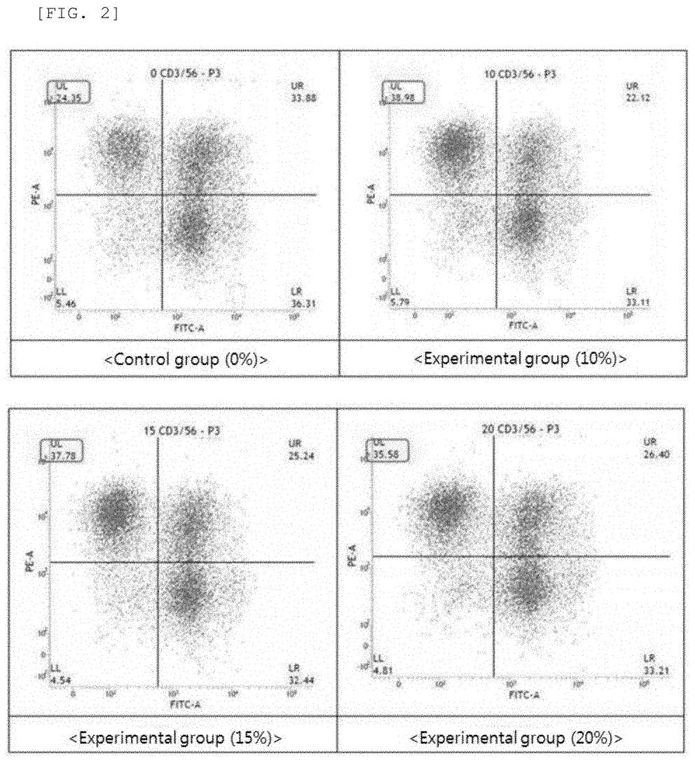

In addition, as shown in FIG. 2 and Table 2 below, it was confirmed that the NK cell fraction was higher in all of the experimental groups in which the healthy-person immune-cell cultured solution was added at concentrations of 10%, 15% and 20% and cultured than in the control group.

TABLE 2

| | NK cell fraction (unit: %) |
|---|---|
| Control group | 24.35 |
| Experimental group (10%) | 38.98 |
| Experimental group (15%) | 37.78 |
| Experimental group (20%) | 35.58 |

### Example 4: Measurement of Cytotoxicity Against Cancer Cells

As an experimental group, immune cells cultured with the addition of a healthy-person immune-cell cultured solution by the method of Example 2 were used, and as a control group, immune cells cultured without the addition of a healthy-person immune-cell cultured solution were used.

Cytotoxicity against cancer cells was determined by mixing K562 cells (Public Health England, 89121407), which are a chronic myelogenous leukemia cell line targeted by NK cells, and immune cells cultured using 10% of a healthy-person immune-cell cultured solution, at ratios of 1:3, 1:5 and 1:10, followed by co-culture using IMDM (Gibco, 12440053) supplemented with 10% FBS for 4 hours.

Only cancer cells were stained with a CFSE solution, which is one component of the 7-AAD/CFSE Cell-Mediated Cytotoxicity Assay Kit (Cayman, 600120), mixed with cells at individual ratios, and then co-cultured using a V-bottomed 96-well plate (Nunc, 249935). After co-culture for 4 hours, dead cells were marked using a 7-AAD solution, which is a kit component. For analysis of the results, 7-AAD-stained dead cells were identified among CFSE-stained cancer cells using a FACS device (BD FACSVerse, BD Bioscience), and the fraction thereof was determined.

Cytotoxicity analysis using the kit was performed following the protocol recommended by the kit manufacturer.

Based on the results thereof, as shown in FIG. 3 and Table 3 below, the cytotoxicity against K562 at respective K562-cell:immune-cell ratios of 1:3, 1:5 and 1:10 was higher in the immune cells (experimental group) cultured with the addition of the healthy-person immune-cell cultured solution than in the immune cells (control group) cultured without the addition of the healthy-person immune-cell cultured solution.

TABLE 3

| | 1:3 | 1:5 | 1:10 |
|---|---|---|---|
| Control group | 4.05 | 6.48 | 14.57 |
| Experimental group (10%) | 31.75 | 37.36 | 52.53 |

Cytotoxicity depending on T:E ratio (unit: %), T = K562, E = immune cell

### INDUSTRIAL APPLICABILITY

According to the present invention, it is possible to increase the rate of proliferation of immune cells including NK cells not only in cancer patients but also in persons who are genetically susceptible to cancer or in elderly people, and to increase the NK cell fraction and cell-killing ability.

Although specific embodiments of the present invention have been disclosed in detail as described above, it will be obvious to those skilled in the art that the description is merely of preferable exemplary embodiments and is not to be construed as limiting the scope of the present invention. Therefore, the substantial scope of the present invention will be defined by the appended claims and equivalents thereof.

1. A method of increasing NK cell content comprising culturing peripheral blood mononuclear cells (PBMCs) derived from a cancer patient or a normal person in a medium containing a normal-person immune-cell cultured solution and a CD3 antibody.

2. The method according to claim 1, wherein the normal person is a disease-free person aged 16 to 40 years.

3. The method according to claim 1, wherein the normal-person immune-cell cultured solution is obtained by subjecting peripheral blood mononuclear cells (PBMC) derived from a normal person to P1-P3 culture and removing cells.

4. The method according to claim 3, wherein the peripheral blood mononuclear cells (PBMCs) are cultured in a medium containing a cytokine antibody selected from the group consisting of CD3, CD16, and CD56.

5. The method according to claim 1, wherein an amount of the normal-person immune-cell cultured solution in the medium is 1-50% (v/v).
